(19) United States
(12) Patent Application Publication
(10) Pub. No.: US 2015/0290639 A1
(43) Pub. Date: Oct. 15, 2015

(54) PRESSURE ASSISTED LATERAL FLOW DIAGNOSTIC DEVICE

(71) Applicant: Vladimir EVTODIENKO, Ottawa (CA)

(72) Inventor: Vladimir Evtodienko, Ottawa (CA)

(21) Appl. No.: 14/648,709

(22) PCT Filed: Dec. 3, 2013
(86) PCT No.: PCT/CA2013/050926

§ 371 Date: May 31, 2015

Related U.S. Application Data
(60) Provisional application No. 61/732,580, filed on Dec. 3, 2012.

(51) Int. Cl. B01L 3/00; G01N 33/543; B01L 7/00

(52) U.S. Cl. CPC B01L 3/50273; B01L 7/52; G01N 33/54386; B01L 2200/10; B01L 2400/0487; B01L 2400/049; B01L 2400/0694; B01L 2300/12; B01L 2300/069; B01L 2300/0825; B01L 2300/0877

(57) ABSTRACT

An external pressure assisted lateral flow diagnostic test device, and method for use thereof, for highly sensitive detection of species of interest. The microfluidic test device comprises a sample and reagent chambers, a detection channel comprising an encapsulated porous membrane with an analyte capture zone, wherein a dynamic pressure causes fluid to flow from the sample and reagent chambers through the detection channel.

### CROSS REFERENCE TO RELATED APPLICATIONS

This application claims benefit of priority to International Patent Application No. PCT/CA2013/050926, filed Dec. 3, 2013, the contents of which are hereby expressly incorporated herein by reference in their entirety and for all purposes.

### FIELD OF THE INVENTION

The present invention pertains to an external pressure assisted lateral flow diagnostic test device, and method for use thereof. The present invention also pertains to a device for analysis of fluids for detecting of chemical and biological species of interest.

### BACKGROUND

There has been great interest in the medical, diagnostic and environmental fields in on site testing for various detectable species. In the medical field, point of care devices that can be used in a physician's office, in the field, or even by patients themselves are very useful. Critical characteristics of any medical diagnostic test includes the time required to get the results from a patient sample, the test sensitivity and specificity. Two common types of device used in on site or point of care testing are lateral flow devices and microfluidic devices. Lateral Flow devices, for example those described in U.S. Pat. Nos. 5,602,040, 5,622,871, 5,656,503, and International published PCT Application publication No. WO 88/08534, are now very commonly used in medical and environmental testing.

U.S. Pat. No. 5,622,871 describes a common geometry for a lateral flow device comprising a porous carrier or strip which can carry a sample to a binding reagent which is immobilized on the strip at the contacting zone. When the sample is contacted with the strip, capillary action causes the fluid to rise through the strip and eventually reach the contacting zone. If a sample contains a species of interest that binds to the binding reagent, exposure of the binding reagent to the species of interest in the presence of a label indicates the presence of the species of interest in the sample. This type of test is widely used in home pregnancy and ovulation tests.

In the field conditions, substances of interest can be found in many different types of media such as, for example, water, effluent, soil, sludge, manure wastes and sediments. Detecting substances of interest in environmental samples is often difficult due to the dilution of the substance of interest. To obtain accurate measurements, samples often have to be taken back to a laboratory for sensitive analysis which can result in a lengthy time delay. One method of detecting low concentrations of hydrophobic analytes in environmental samples is described in U.S. Pat. No. 5,891,740. Systems for detection of many different types of substances at low concentration without requiring complex or sensitive laboratory equipment are very desirable for environmental detection in the field.

Despite the widespread acceptance of this type of lateral flow technology, these devices have several limitations which include slow development of signals, poor sensitivity, and poor limits of detection for rapid tests. As a standard lateral flow assay depends on capillary force, the detection time is limited by the surface tension of the carrier fluid. In addition, there is often a low signal/noise ratio due to the lack of a washing step, and often a low limit of detection due to the limited sample volume, which is limited by slow sample movement through the absorbent developing medium (e.g., the porous membrane strip) driven by capillary forces.

Another widespread test format for rapid testing is the use of microfluidic devices. Microfluidic devices can be used for a variety of clinical and chemical tests such as immunoassays (see for example, U.S. Pat. No. 4,775,515; Hatch, A. et al. 2001 Nature Biotechnology 19, 461-465; Eteshola, E. & Leckband, D. 2001 Sensors and Actuators B-Chemical 72, 129-133; Cheng, S. B. et al. 2001 Analytical Chemistry 73, 1472-1479; and Yang, T. L. et al. 2001 Analytical Chemistry 73, 165-169), flow cytometry (see for example U.S. Pat. No. 6,713,298; and Sohn, L. L. et al. 2000 PNAS 97, 10687-10690), PCR amplification (see for example Belgrader, P. et al. 2000 Biosensors & Bioelectronics 14, 849-852; Khandurina, J. et al. 2000 Analytical Chemistry 72, 2995-3000; Lagally, E. T. et al. 2001 Analytical Chemistry 73, 565-570; and Buchholz, B. A. et al. 2001 Analytical Chemistry 73, 157-164), and DNA analysis (see for example Buchholz, B. A. et al. 2001 Analytical Chemistry 73, 157-164; Fan, Z. H. et al. 1999 Analytical Chemistry 71, 4851-4859; Koutny, L. et al. 2000 Analytical Chemistry 72, 3388-3391; and Lee, G. B. et al. 2001 Sensors and Actuators B-Chemical 75, 142-148).

Microfluidic devices can move liquids through the system by various means including capillary action (U.S. Pat. No. 4,775,515), gravitational force (U.S. Pat. No. 5,225,163), centrifugal force (WO 2009/39239 A2), hydrostatic pressure (D. L. Stokes et al. 2001 J. Anal. Chem. Vol. 369 p. 295) and electrokinetic flow (U.S. Pat. No. 7,214,300). Some of the advantages of microfluidic devices include the ability to mix reagents during the assay and to wash immobilized substances to remove excess reagents and interfering substances. These advantages contrast with lateral flow devices where mixing and washing are difficult to achieve.

Some pressure actuated devices, such as that described in U.S. Pat. No. 8,124,026 use electro-osmotic pumps to push liquids through a laminated lateral flow element for the purpose of quantitative assay. However, fabrication of these devices requires complicated integration of electrical and microfluidic circuits. Further, electro-osmotic pumps are restricted to pumping only very diluted electrolytes and usually not capable of direct pumping of the sample and/or ready to use reagent solutions.

Another pressure actuated device is described in United States Patent Application Publication Nos. 2009/0181411 and US 2012/0164627. These publications describe a device having a reagent strip with multiple reagent zones wherein liquids are pushed inside reagent strip from reagent compartments with air-pressurized actuators. The reagent strip described is a void volume with solid phase capture means incorporated into the flow channels, and the solid phase can be a surface, beads or even porous adsorbent placed into the assay chamber. However, these publications do not teach a porous membrane material to be encapsulated or sealed or laminated into their flow path. Rather, in this case, sample flows along the surface of the adsorbent solid phase material with no tight sealed to ensure that no flow can bypass membrane pores or the analyte capture species while the device is under pressure.

In recent years microfluidic devices have become an integral part of an assay platform designed as lab-on-chip test systems. However, these micro-fluidics cartridges are often very sophisticated devices, and/or are fabricated with expensive materials and technologies. There remains a need for a simple, inexpensive test capable of high sensitivity, low detection limit, and a high degree of accuracy.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an external pressure assisted lateral flow diagnostic test device, and method for use thereof, for highly sensitive detection of species of interest, wherein a dynamic pressure causes fluid to flow from the sample and reagent chambers through the detection channel.

In accordance with one aspect, there is provided a device for detecting an analyte of interest in a sample, wherein the device is a microfluidic cartridge comprising: a sample chamber for receiving the sample; at least one secondary fluid chamber; at least one detection channel fluidly connected to said sample chamber and said at least one secondary fluid chamber via one or more microfluidic channels, wherein each of said at least one detection channels comprises an encapsulated porous membrane strip having at least one analyte capture zone comprising a plurality of immobilized capturing species; and means for generating a dynamic pressure between the sample chamber and the at least one detection channel to facilitate fluid to flow from the sample chamber through each of the at least one detection channels and to increase the volume of fluid that can flow through each of the at least one detection channels over the volume of fluid that can flow through each of the at least one detection channels when no dynamic pressure is applied.

In accordance with one embodiment, the sample chamber and/or the secondary fluid chamber has a flexible cover, flexible bottom and/or flexible walls.

In accordance with another embodiment, the sample chamber and/or the secondary fluid chamber is formed by a rigid base layer of the cartridge.

In accordance with another embodiment, the dynamic pressure is generated by pressure applied to the sample chamber. In one embodiment, the pressure applied is applied to the sample chamber by a mechanical actuator. In one preferred embodiment, the pressure applied is between about 5 psi and about 300 psi, or between about 5 psi and 100 psi.

In accordance with another embodiment, the detection channel is formed with adhesive or thermoplastic film with high physical adhesion to the porous membrane.

In accordance with another embodiment, the dynamic pressure is generated by a vacuum applied to the downstream end of the detection channel.

In accordance with another embodiment, the secondary fluid chamber is connected to the microfluidic channel.

In accordance with another embodiment, the device further comprises a waste chamber at the downstream end of the detection channel. In one preferred embodiment, the waste chamber comprises a stop flow valve, more preferably the stop flow valve is covered by a membrane which is permeable to gas and impermeable to fluid. In another preferred embodiment, the stop flow valve has an adaptor to receive a vacuum.

In accordance with another embodiment, the sample chamber comprises a reagent pad. In one preferred embodiment, the reagent pad comprises analyte specific antibodies, buffers, enzyme labelled antibody conjugates, or optionally labelled capture species. In another preferred embodiment, the reagent pad comprises release agents and/or stabilizers.

In accordance with another embodiment, the immobilized capturing species is an antibody that can specifically bind the analyte of interest. In one preferred embodiment, the immobilized capturing species is streptavidin.

In accordance with another embodiment, the device comprises more than one detection channel. In another embodiment, the detecting channel is formed between layers of the cartridge.

In accordance with another embodiment, the secondary fluid chamber is pre-filled with washing buffer. In accordance with another embodiment, the secondary fluid chamber is pre-filled with enzyme substrate. In accordance with another embodiment, the enzyme substrate is a chromogenic, a fluorescent or a chemiluminescent substrate.

In accordance with another embodiment, the device comprises more than one secondary fluid chamber. In accordance with another embodiment, the cartridge is disposable.

In accordance with another embodiment, the sample chamber, the detection channel or both can be heated to a predetermined temperature or temperature range. In one preferred embodiment, the heating is 25° C. to 50° C. or about 37° C.

In accordance with another embodiment, the volume of fluid flowing through the detection channel when the dynamic pressure is applied is 2 to 200 times, and preferably 10 times greater than the volume of fluid flowing without the applied dynamic pressure.

In accordance with another embodiment, the cartridge is made from the two or more layers of pharmaceutical blister packaging materials and/or plastic molded parts. Preferably, the pharmaceutical blister packaging materials comprise metal foils or multi-layer plastic films, optionally with water vapour and oxygen barrier properties.

In accordance with another embodiment, the porous membrane has a pore size from about 0.1-20 microns, and preferably 0.45-5 micron. In another embodiment, the microfluidic channel connects the secondary fluid chamber to the detecting channel.

In accordance with another aspect, there is provided a method of detecting an analyte in a fluid sample, the method comprising: (a) introducing a sample into a sample chamber with a plurality of detecting species under conditions suitable for formation of complexes between the analyte and detecting species; (b) injecting the sample comprising the complexes formed in step (a) onto a porous membrane having an analyte capture zone with a plurality of immobilized capturing species; and (c) detecting the presence of the analyte by detecting complexes immobilized at the analyte capture zone, wherein the step of injecting the sample is carried out by generating a dynamic pressure between the sample chamber and the detection channel, and wherein the sample chamber and the detection channel are fluidly connected by a microfluidic channel in a cartridge.

In accordance with one embodiment, the dynamic pressure is generated by pressure applied to the sample chamber. In accordance with another embodiment, the pressure applied is applied to the sample chamber by a mechanical actuator. In one preferred embodiment, the pressure applied by the mechanical actuator the pressure applied by the mechanical actuator is between about 1 psi and about 300 psi, preferably between about 5 psi and about 100 psi.

In accordance with another embodiment, the dynamic pressure is induced by a vacuum applied to the downstream end of the detection channel.

In accordance with another embodiment, the injecting step takes from between about 1 to 60 minutes, preferably between about 10 and 20 minutes. In accordance with another embodiment, the sample comprises water, a water based extract, or a biological fluid.

In another embodiment, the method further comprises the step of detecting species from the capture zone in the detection channel prior to step (c), wherein the step of washing unbound detecting species comprises generating a dynamic pressure between the secondary fluid chamber and the detection channel. In one preferred embodiment, the washing step takes between about 10 seconds and about 300 seconds, preferably less than 60 seconds.

In accordance with another embodiment, the volume of sample flowing through the detection channel when the dynamic pressure is applied is 10 to 100 times greater than the volume of sample flowing through the detection channel in the absence of the applied dynamic pressure.

In accordance with another embodiment, the cartridge is made from two or more layers of pharmaceutical blister packaging materials and/or plastic molded parts.

In accordance with another embodiment, the detecting step is carried out using a signal-detecting instrument. In one preferred embodiment, the detecting species comprises a conjugated enzyme and the detecting step comprises injecting enzyme substrate into the detection channel. In another embodiment, the enzyme converts the enzyme substrate to a detectable product.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is a top plan view of one exemplary device having a sample chamber, a wash chamber and a secondary fluid chamber;

FIG. 2 is an alternative top plan view of one exemplary device having a sample chamber, a wash chamber and a secondary fluid chamber;

FIG. 3 is a cross sectional view one embodiment of the device through the line A-A shown in FIGS. 1 and 2;

FIG. 4 is a cross sectional view one embodiment of the device through the line A-A shown in FIGS. 1 and 2;

FIG. 5 is a cross sectional view one embodiment of the device through the line A-A shown in FIGS. 1 and 2;

FIG. 6 graphically depicts the light generation and luminometer response for variable volumes of biotinylated alkaline phosphatase; and

FIG. 7 graphically depicts the effect of the C. difficile toxin A concentration on light generation and luminometer response.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “sample fluid” encompasses any liquid or fluid sample that may contain the chemical or biological species of interest. The sample fluid can be a liquid, solution, suspension, emulsion, complex mixture, or a buffer which has been mixed with a non-liquid or liquid sample. Some examples of sample fluids include whole blood, bacterial cell suspensions, or mixtures into buffer of body fluids. This can also include environmental fluids such as water from any source, or solid samples dissolved in water or buffered water.

As used herein, the term “body fluid” can include amniotic fluid, bile, blood, serum, breast milk, cerebrospinal fluid, lymph, feces, ejaculate, mucous, pus, saliva, sweat, tears, secretions, vomit and urine.

As used herein, the term “microfluidic channel” refers to a channel that connects the chambers and elements of the device, wherein the maximum interior diameter of the channel is about 1 mm or less than about 1 mm. A “microfluidic device” has at least one microfluidic channel.

As used herein, the terms “upstream” and “downstream” refer to the locations within the device as they related to the direction of fluid flow, which is from the upstream end to the downstream end of the device. Specifically, fluid must flow from the sample chamber (upstream end of the device) through the porous membrane and the analyte capture zone (downstream end of the device).

The term “dynamic pressure,” as used herein, refers to the pressure gradient that is generated as a result of pressure applied to the upstream end of the device or vacuum applied to the downstream end of the device such that the pressure of the fluid flow through the porous membrane in the detection channel is increased. It is understood that application of pressure at the upstream end of the device can induce a similar pressure gradient as can be induced by application of reduced pressure or vacuum at the downstream end of the device.

As used herein, the term “pressure actuator” refers to any means by which the pressure inside the device is changed to enable the fluid flow from the upstream end of the device to the downstream end of the device. The pressure actuator can be any component system or apparatus that applies increased pressure at the upstream end of the device, or that applies a vacuum or reduced pressure at the downstream end of the device.

As used herein, the term “porous membrane” refers to the fluid permeable porous material that can serve as a sample flow pass and can contain at least one or multiple analyte capture zones, as described in more detail below. The porous membrane used in the present device is an encapsulated porous membrane. The term “lateral flow membrane” (LFM) is another term describing the porous membrane strip which contains at least one or multiple analyte capture zones. In the case of standard rapid assays, lateral flow usually means sample fluids flow along the porous membrane driven by capillary forces.

As used herein in referring to an assay, such as a diagnostic assay, the term “sensitivity” refers to the measurement of the proportion of actual positives in the assay which are correctly identified as such. The term sensitivity should not be confused with the term “limit of detection” or “analytical sensitivity”, which is the lowest concentration of analyte that can be accurately measured.

As used herein, the term “specificity” refers to the measurement of the proportion of negatives which are correctly identified. In other words, specificity is a measure of the proportion of false positives obtained.

The present pressure assisted lateral flow device comprises a cartridge having a sample chamber and fluidly connected detection channel having an encapsulated porous membrane element comprising an analyte capture zone connected to the sample chamber via at least one microfluidic channel. An externally applied upstream pressure or downstream vacuum forces sample fluid from the sample chamber through a microfluidic channel to the upstream end of the detection channel and through the porous membrane in the detection channel to the downstream end of the detection channel. This configuration allows large sample volumes to pass through the porous membrane and analyte capture zone so that immunoassay results can be obtained on samples of varying analyte concentration, which may be very low concentration, with good accuracy and high sensitivity and within a reasonable time frame for point-of-care assays.

A dynamic pressure established between the sample chamber and the porous membrane causes fluid to flow through the device and, consequently causes any complex formed between a detecting species and the analyte of interest to contact the analyte capture zone and become immobilized at the capture zone. In this way, the applied dynamic pressure establishes a fluid flow in the direction from the sample chamber through the detection channel and the porous membrane where the analyte of interest can be detected as a result of immobilization at the analyte capture zone. The dynamic pressure allows large sample volumes to pass through the detection channel within a reasonable time frame. As a result, larger volumes of fluid can be analysed and more analyte concentrated on the capture zone within detection channel than is possible using prior art devices. This can significantly increase the sensitivity specific signal of assay without increasing non-specific binding and improve signal to noise ratio. This can result in higher immunoassay analytical sensitivity than standard rapid capillary flow assays.

The device can be adapted to allow generation of a dynamic pressure either from application of external pressure via a pressure actuator at the fluid chamber(s) and/or application of a vacuum via a pressure actuator at the downstream end of the detection channel. The components within the present device must be able to withstand the increased internal pressure generated by the pressure actuator(s). In one embodiment, the present device can be operated with a variable internal pressure, preferably between about 5 psi and about 100 psi.

Sample volumes ranging from a few microlitres to millilitres can be applied to the sample chamber and forced through the analyte capture zone within the detection channel. The term ‘forced’ refers to the direction and dynamic pressure of fluid flow as a result of the applied pressure or vacuum to the device. As the volume of sample can be 10 to 100 times larger than standard capillary forces driven assay for the same porous membrane strip geometry, the amount of analyte captured and concentrated at the analyte capture zone is also relatively higher. This can allow for a relative and significant improvement in assay sensitivity compared to standard lateral flow assays that do not have an externally applied pressure or vacuum source.

The detecting species can be a labelled or conjugated antibody, receptor, or any other molecules capable of binding selectively to the analyte of interest and of being directly detected (e.g., via a chromophore, flurophore or chemiluminophore label) or indirectly detected (e.g., via secondary means such as binding to a labelled, secondary antibody). The detecting species can also be a labelled or conjugated secondary antibody capable of binding selectively to a primary antibody that binds selectively to the analyte of interest. This detecting species can be, for example, an antibody bound directly to a chromophore, fluorophore, radiolabel, or enzyme. Various enzymes are known to be used as detecting species conjugates, for example alkaline phosphatase (AP), horseradish peroxidase (HRP) or any other enzyme label known in enzyme-linked immunoassay. The antibody can also be a primary antibody that is conjugated to molecule that can be specifically recognized a secondary binding agent. This detectable species can be, for example, an antibody bound to biotin, which can specifically recognized and bound by a labelled streptavidin.

The detecting species binds to the analyte of interest to form a complex. The complex is then immobilized on the analyte capture zone in the porous membrane and detected by a detector. The detecting species is any species that binds to the analyte of interest which can then either be detected by the detector directly, or can convert a substrate into a detectable molecule or mixture. In the case where the detecting species is an enzyme linked antibody, the enzyme can react with the enzyme substrate to produce a detectable colorimetric, chemiluminescent or fluorescent signal.

The enzyme substrate or label for the detecting species can be one or more colorimetric, fluorescent or chemiluminescent enzyme substrates. Some chemiluminescent substrates for HRP include Luminol™ from Aldrich Chemical Co, Femtoglow™ from Michigan Diagnostics, TMA-6, TMA-3, PS-3 or PS-atto from Lumigen. Some chemiluminescent substrates for AP include CDP-Star™ and CSPD from Tropix or AP substrate from Michigan Diagnostics. Ready to use 3,3′,5,5′-tetramethylbenzidine (TMB) solution can also be used as colorimetric HRP substrate.

To introduce the detecting species to the sample, the sample chamber or secondary fluid chamber can contain liquid or dried reagent components. These reagent components can include but are not limited to the detecting species, biotinilated capture antibody conjugates, buffers, release agents, and stabilizers. The reagent components can be dried directly in the sample chamber or on reagent pad. The reagent pad can utilize any porous matrix with low protein binding capacity such as polyester or glass fibre. For example the fibre can be made from GF/DVA, MF1 from Whatman or any other conjugate release matrix from Pall, Ahlstrom or GE. The reagent pad can also comprises buffers, preservatives, release agents and/or stabilizers. The dry reagent pad ca also contain carbohydrate based stabilizers such as but not limited to sucrose, trehalose, sorbitol, lactitol, dextran, and mixtures thereof. The dry reagent pad can also contain protein based stabilizers and release agents such as but not limited to bovine serum albumin, polymer based stabilizers, release agents such as but not limited to polyvinyl alcohol, detergents or surfactant based release agents such as but not limited to Triton X-100, Tween 20, and mixtures thereof.

Alternatively, the sample chamber or a secondary fluid chamber can contain a liquid mixture of detecting species secondary enzyme labelled antibodies, or labelled antibodies, primary biotin labelled antibodies and/or enzyme substrate. Alternatively, but perhaps less conveniently, the antibody conjugates and the sample can be mixed externally and then the reacted sample added to the sample chamber. In one alternative, the sample chamber can simply be a sample entry port for injecting a sample into the microfluidic channels. Prior to use of the detection device, the reagents in second chamber need to be in liquid form, or reconstituted by the addition of liquid.

The detection channel comprises an encapsulated porous membrane that allows passage of fluid, and prevents fluids from bypassing the porous membrane, for example along the inner surface of the detection channel. In this way, all fluid material introduced at the upstream end of the detection channel is forced to pass through the porous membrane and the analyte capture zone on its way to the downstream end of the detection channel. The porous membrane has pores of sufficient size to allow the flow of the sample solution through the detection channel.

In one embodiment, the detection channel can be formed by layers of plastic material or matrix during encapsulation of the porous membrane during manufacture of the device. The membrane encapsulating material can thus form an air and liquid impermeable barrier channel around the porous membrane. The membrane encapsulating plastic material can also have strong physical adhesion to the porous membrane material under the dry and wet contact. Preferably, the encapsulated porous membrane can withstand up to at least 50 psi and up to about 300 psi, or up to about 100 psi, of external pressure without breaking the capsule or delaminating and forming leaks along the detection channel surface.

The porous membrane can comprise any porous material that enables liquids to pass through it under pressure while substantially maintaining its structural integrity. Some examples of such porous materials are nitrocellulose, mixed esters cellulose, polyester, nylon ore any other porous material. Pore sizes of from 0.1 to 50 micron can be used, with membrane pore sizes of from 0.45 to 5 microns preferred. Membranes such as Biodyne™ A or B, Loprodyne™ or Immunodyne™ from Pall, or Osmonics™ Nylon from GE can be used. Other membranes that can be used include nitrocellulose membranes with pores 0.1-100 micron from Millipore, Whatman, Pall, or GE. The width of the porous membrane can be between 0.5 and 5 mm, with 1 to 2 mm width preferred. It is understood that the interior diameter of the detection channel will be the same as the width of the porous membrane and also encapsulating plastic material can have good physical adhesion to the porous membrane material to prevent liquid from bypassing the porous membrane and the analyte capture zone.

The porous membrane has an analyte capture zone that comprises a capturing species immobilized in the capture zone through the physical adsorption or covalent binding to the porous matrix. The capturing species is immobilized onto the analyte capture zone to capture any analyte of interest or antibody conjugated to the analyte of interest that passes through the detection channel. The capturing species can be one that directly binds to the analyte of interest, such as an analyte specific antibody. One example of capturing species of this type is one used in a sandwich-type Enzyme-linked immunosorbent assay (ELISA), in which an antibody for the analyte of interest is bound to the analyte capture zone and a detecting species that also binds to the analyte of interest is present to enable detection of the presence of the analyte. In this example, the capture antibody binds to a different epitope on the analyte of interest than the detecting species (or antibody). Alternatively, the capturing species can bind to a detecting species that has complexed with the analyte of interest. An example immunoassay of this type is a biotin-streptavidin binding assay wherein the streptavidin (capturing species) is immobilized on the porous membrane at the analyte capture zone, and the biotin (detecting species) is conjugated to an antibody which binds the analyte of interest. In either case, the presence of the detecting species at the analyte capture zone is indicative of the presence of the analyte of interest in the sample.

The present device can also have more than one (multiple) detection channel for detecting multiple substances of interest in the same test. The multiple detection channels can be arranged in parallel, wherein the microfluidic channel originating from the sample chamber is split into multiple channels for directing the sample fluid to the multiple detection channels. Alternatively, the multiple detection channels can be arranged in series such that the effluent from one detection channel is connected to the upstream end of the next detection channel in the series.

The present device can also have more than one (multiple) capture zones within the same detection channel for detecting multiple substances of interest in the same test or for the negative test results control purposes (control zone). The multiple capture zones can be arranged in sequence one after another on the porous membrane.

As noted above, to force the fluid flow from the sample chamber through the detection channel, the device must be able to receive an external pressure and/or vacuum. In one example, the device is configured to receive an external pressure by the fluid chamber(s), including the sample chamber, having at least one flexible wall for receiving pressure for a mechanical pressure actuator. The fluid chamber(s) can have a flexible cover layer, flexible chamber bottom, flexible chamber walls, or any combination thereof. In this way, sufficient pressure applied to the fluid chamber top, bottom or walls, respectively, will force the fluid from the chamber, through the microfluidic channel(s), and into the detection channel. Various types of mechanical pressure actuators can be used with the device. For example, an automated sample injection system can be used to apply pressure to a surface. Sufficient pressure can also be obtained by compression of the fluid chamber(s) with thumb(s) and/or finger(s).

Alternatively a vacuum source can be applied to the downstream end of the detection channel that will establish a fluid flow from the fluid chamber(s). A device for use with a vacuum can also be fitted with an adaptor to receive the vacuum, and, optionally, a barrier membrane between the fluid flow region and the point of attachment to the vacuum so that fluid will not be drawn into the vacuum system. In one particular case, the barrier membrane can be a porous hydrophobic membrane. When the device has a waste chamber, the vacuum attachment is preferably on a surface of the waste chamber.

The device can also optionally comprise one or more secondary fluid chamber(s) comprising one or more detecting species, secondary detectable species, or wash solution or enzyme substrate. As described above, the device can comprise ready to use reagents, for example for a complete rapid immunoassay test. The secondary fluid chamber(s) can also be empty for receipt of any fluid desired by the user or can comprise water or buffer for use as a wash solution. When the device has more than one fluid chamber, the chambers can be pressure-actuated simultaneously or sequentially. When the dynamic pressure in the device is actuated by compressing the sample and/or fluid chambers, pressure can be selectively applied to each chamber to obtain the desired sequence of application of each liquid sample. The device further comprises breakable liquid and gas-tight membranes, or valves, between the fluid chamber(s) and the microfluidic channel(s) upstream from the detection channel that can be broken to release the contained liquid into the detection channel. In the case where the device comprises a wash chamber, the wash fluid can wash any unbound detector species (or enzyme label) from the analyte capture region to improve the assay results sensitivity by reducing non-specific binding and increasing signal to noise ratio.

The device can optionally further comprise a waste chamber that collects fluid from the downstream end of the detection channel. The sample waste compartment can collect sample, wash buffer and other fluids which pass through the porous membrane in the detection channel. The waste chamber is connected to the porous membrane in such a way as to allow any fluid exiting the detection channel to be collected.

The waste chamber outlet can further have a stop flow valve covering the waste chamber outlet. The stop flow valve can be a membrane that is permeable to gas but impermeable to liquid, thereby allowing the venting of air as liquid fills the device, while trapping the liquid. The stop flow valve can be a hydrophobic membrane or porous semi-permeable hydrophobic membrane, which allows the venting of air as liquid fills the device while trapping the liquid. For example the stop flow valve can be made from Versapor™ 800R membrane from Pall or any other hydrophobic air permeable porous material can be used for this purpose. The waste chamber can also comprise an outlet configured to receive a vacuum, as discussed above. The waste chamber can also hold a fixed volume of waste.

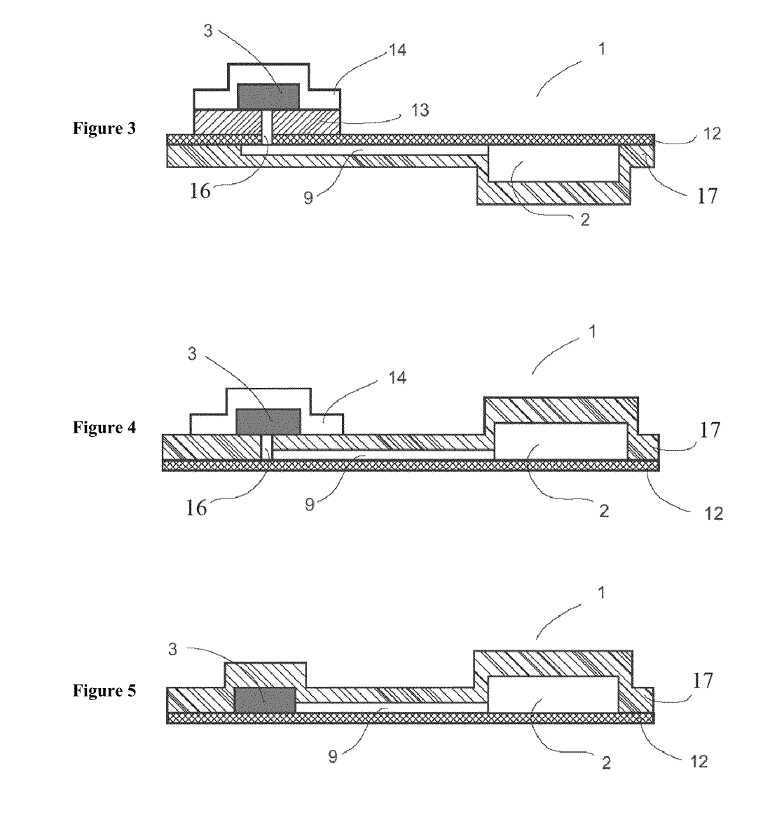

Various configurations of exemplary cartridges are shown in FIGS. 3, 4 and 5. The cartridge which supports the present device can be made of any material that can be formed to accommodate the described elements. In one example, the cartridge consists of at least two layers:
1. a rigid cartridge base layer (11) made of plastic film(s) thermoformed to a specific three-dimensional shape
2. a flexible cover layer (12) which covers the chamber(s) and
   The cartridge also optionally has a binding adhesive film layer cut to a specific shape, to bind the base layer to the cover layer, between the base layer (11) and the flexible cover layer (12). The binding layer can be made from single or double-sided pressure sensitive adhesive tape with thermoplastic heat activated adhesive film(s).

The cartridge can also optionally comprise an additional top encapsulation layer (14) for encapsulating the porous membrane, and a further optional base membrane layer (13). The assembled layers form system of channels and cavities to allow for the lateral flow porous membrane and liquid reagent encapsulation. Alternatively the cartridge components, specifically the base layer (11), can be made and assembled from plastic moulded parts.

The cartridge base forming layers used for manufacture of the cartridge can be made of any plastic sheet material. Some examples of these are polycarbonate, polyester, polystyrene or similar material. Preferably, pharmaceutical-grade blister packaging materials with oxygen and water vapour barrier properties can be used. Rigid thermoforming or cold forming multi-layers films for medical packaging such as those made from Aclar™ VapoShield™ polymer film or thick and rigid aluminum foil are very suitable for cartridge forming layer application. One especially desirable film for this purpose is multi-layer medical packaging film. In one example, VPOA 10200 film from TekniPlex™ can be used. Cartridge base layers can be pressed or heat-sealed together, or assembled in any other way known to the skilled person. The cartridge can also be disposable.

The flexible cover layer (12) can be made from any flexible plastic sheet material. Flexible multi-layers films for medical packaging were is one of the layers made from Aclar, VapoShield polymer film or flexible aluminum foils are very suitable for the flexible cover layers application. For example, Teknilid™ PSPET multi-layer foil from Tekniplex can be used as flexible cover. Binding layers can be made from single or double-sided pressure sensitive adhesive tapes with silicon, acrylic or rubber based adhesives, and thermoplastic films made polyethylene, polyurethane or any other heat activated adhesive material.

The detection channel can be formed by the layers of plastic material which has strong physical adhesion to the porous membrane material under the dry and wet contact and can thus form an air and liquid impermeable barrier channel around the porous membrane. The membrane encapsulating plastic material can be chosen from any low melting temperature thermoplastic films such as ethylene, nylon or low melt temperature adhesive films. For example, thermoplastic adhesive films from Adhesive Research can be used for porous membrane strip encapsulation purposes.

Cartridge layers and detection channel can be pressed or heat-sealed together, or assembled in any other way known to the skilled person. Alternatively, the detection channel can be formed inside the cartridge between the base layer and the flexible cover layer. In another alternative, the flexible layer and the binding layer can be integrated in a single layer. In this way, the device can comprise only two layers.

The presence of the analyte of interest at the analyte capture zone can be determined via a signal detection instrument or by visual observation. The signal detection instrument or detector may be simply a transparent cover over the capture region that allows the presence of analyte to be determined visually. The detector may also be a more complex instrument to determine the presence of analyte in the capture region by a qualitative or quantitative instrumental method. Such methods can include measurement of optical absorbance, chemiluminescence, fluorescence, electrical potential and amperometry. The signal detection instrument is preferably a light sensitive diode detector, photo multiplier, or light image capture device such as CCD camera and a transparent cover over the analyte capture zone that allows reading and/or detection therethrough. The signal detecting instrument can further comprise a device (cartridge) heating element to increase immunoassay kinetics, reduce analyte capture time and/or increase assay sensitivity, and/or can be used to adjust the temperature in the detection channel to an optimum, or near optimum, temperature for activity of an enzyme label, and/or can be used to adjust the temperature in the detection chamber to improve wash efficiencies.

Method of Use

In using the present device, a sample is loaded onto the device via the sample chamber. The sample can be loaded into the sample chamber as is, or it may be pre-prepared by mixing with liquids such as buffers, water, or other solvents that are required for the specific assay. The sample chamber can also be pre-filled with one or more fluids such as buffers, water or solvents, as well as one or more detectable species. The sample can be water, a water based extract, or any biological fluid. The sample can also be a semi-solid or may have solid inclusions, in which case a filter can be used at the exit of the sample chamber to prevent solids from entering the detection channel. In the case when the device is being used for an immunoassay, the sample can be exposed to an antibody either prior to application into the sample chamber, or in the sample chamber itself.

If the analyte of interest is present in the sample, an analyte-antibody complex will form. An optional incubation period prior to injection of the sample into the detecting chamber may enable more present analyte to bind to the detectable antibody or detecting species. Prior to the application of pressure or vacuum to the device, the sample can be optionally incubated in the sample chamber with or without heating. The sample can be heated, for example from 25° C. to 50° C. An incubation temperature of about 37° C. is preferred. An incubation period of from about 5 to about 60 minutes is preferable, and a less than 10 min incubation period is more preferable. Alternatively, the incubation step can be avoided, and any analyte-antibody binding can take place during sample injection step.

To inject the fluid sample, either pressure or vacuum is applied to the device to establish the dynamic pressure, as described above. In operation, fluid is applied from the sample chamber (sample fluid) and optionally from any additional fluid chambers, as desired. The sample fluid, as well as one or more detecting species and optionally a capturing species, is forced through the microfluidic channel(s) to a detection channel which comprises a porous lateral flow membrane, or porous membrane. The sample and the one or more detecting species and optionally the capturing species can originate in the same sample chamber, or one or more detecting species or the species can be stored in one or more wash chambers or secondary fluid chambers. The washing fluid and the enzyme substrate can be held in separate fluid chambers for simultaneous or sequential addition to the sample chamber or the detection channel or can be combined in one solution for a single injection.

The sample moves through the porous membrane inside the detection channel by the dynamic pressure. Preferably, the sample injection step takes from 1 to 60 minutes, with a more preferable injection time of between 10 and 20 minutes. Of course, the exact time required to inject the sample will be determined by the pressure applied or vacuum applied, flow rate of the fluid, and volume of the sample chamber. The amount of sample pushed through the membrane can be as little as a few microlitres and up a few millilitres. In one specific example, a sample volume of between 20 and 100 microlitres can be forced through a detection channel with an interior cross-section of 0.1-0.2 square millimetres. Ultimately, the sample comes into contact with the analyte capture zone inside of the detection channel, which comprises a capturing species. The capturing species will bind the analyte of interest or analyte complexed with detecting species as described. Detection channel can be heated from 25° C. to 50° C. to accelerate kinetics of analyte capture. Preferably, a heating temperature of 37° C. is preferred.

In the case where the device comprises a wash chamber, the fluid from the wash chamber is injected into the detection channel after the sample fluid. This is accomplished either in a positive pressure system with applied pressure via second mechanical actuator which pressurizes the wash chamber, or in a negative pressure system with vacuum by sucking the wash fluid from the wash chamber. The wash chamber can have a liquid and gas-tight seal which can be broken when injection of the wash fluid is desired. A typical wash step takes between about 30 seconds and about 300 seconds. A wash time under 60 seconds is preferred. In an immunoassay, a wash step can separate specifically bound detecting species such as enzyme labelled antibody complexed with the analyte and free enzyme labelled antibody conjugate in capture zone and reduce non-specific binding.

In the case where the device comprises a secondary fluid chamber, the fluid from this chamber can be injected into the detection channel at a desirable time. Like the injection from the sample chamber and wash chamber, this can be accomplished either by positive pressure applied to the chamber, or negative pressure applied at the downstream end of the detection channel. In an immunoassay, this secondary chamber can be used to house enzyme substrate. Chemiluminescent enzyme substrate will react and generate light that can be detected by the analyzer. The light intensity is proportional to the amount of complex captured in the reaction zone and the concentration of analyte in the sample.

The light intensity can then be measured by light sensitive diode or photo-multiplier. Alternatively, image of the detection channel can captured by CCD camera and analyzed by ImageJ™ or other custom image analyzing software. Alternatively, the entire injection procedure described and signal detection and analyzing process can be carried out by an automated analyzer with single device, or by multiple devices adapted to perform the above-described steps. Alternatively, in the case of colorimetric, fluorimetric, or radiolabelled enzyme substrate, other known detection methods can be used.

Example Device

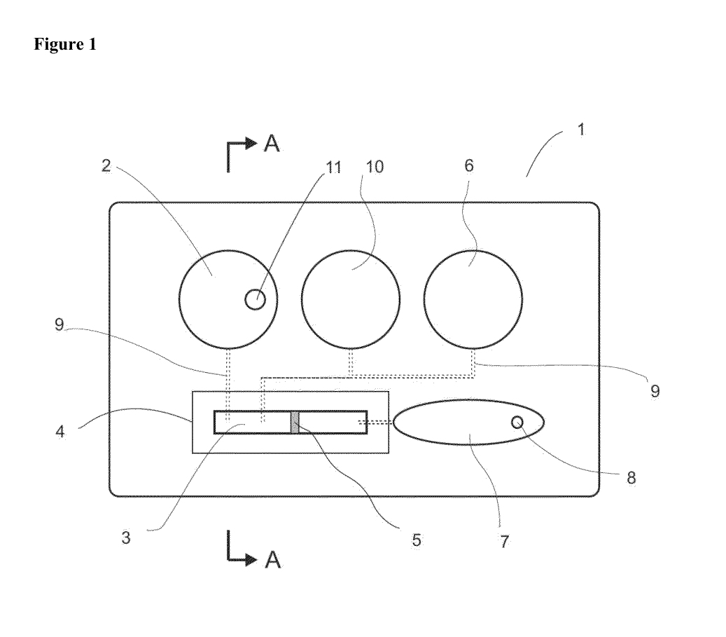

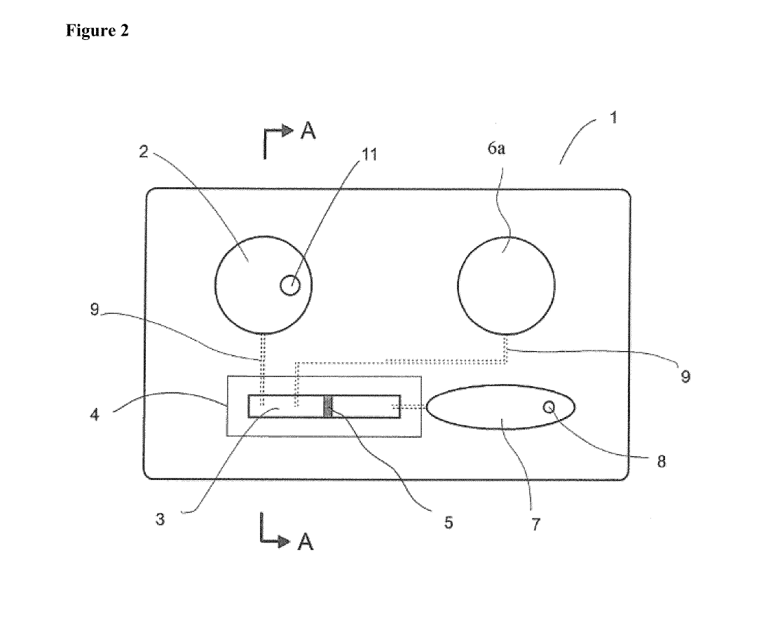

One exemplary device is shown in FIG. 1. A cartridge (1) is shown, which houses the present device. The device comprises a sample chamber (2) having a sample entry port (11), and a detection channel (4) with an encapsulated lateral flow porous membrane (3). The lateral flow membrane (3) has an analyte capture zone (4). Inside the porous membrane is an analyte capture zone (5) to which is immobilized a capturing species. Also provided is a wash chamber (10) and secondary fluid chamber (6). Also provided is a waste chamber (7) having a waste chamber port (8). A system of micro-fluidic channels (9) connects the different fluid chambers to the detection channel (3). As shown in FIG. 2, the wash chamber and secondary fluid chamber may sometimes be combined into a single element or alternate wash chamber (6a). In this embodiment, the other elements of the device are similar to described with respect to FIG. 1.

In the embodiment shown in FIG. 1, a first pressure actuator pressurizes the sample chamber, for example through the flexible cover layer, and pushes the fluid sample through the micro-channel system and onto the porous membrane in the detection channel. After the sample is added, the sample chamber should be sealable to allow the application of pressure to the device which causes the sample to move through the micro-fluidic channels and the membrane. A second pressure actuator pressurizes the wash chamber and third actuator pressurizes the secondary fluid chamber, for example through the flexible cover layer, and pushes the fluid sample through the micro-channel system and onto the porous membrane in the detection channel. In an alternative embodiment, the device can be operated by vacuum downstream the detection channel, and the dynamic pressure can be applied by breaking a membrane seal at the connection point between the liquid chambers 2, 10, 6 or 6a and the micro-fluidic channels to which they are fluidically connected.

A sample application port (11) is provided to enable the addition of sample to the sample chamber. The sample application port is sealed prior to applying pressure to sample chamber. Sealing can be accomplished by pressure adhesive cap, rubber cap, plastic cap, or any other sealing method known to the skilled person.

The cartridges shown in FIGS. 3-5 are different configurations of embodiments of the device as presently described. Specifically, FIGS. 3, 4 and 5 are cross-section views of the device on FIG. 1 at line A-A in FIG. 1. Cartridge (1) is assembled with a combination of several layers. Cartridge base layer (17) Cartridge (1) shown is formed using made from rigid layers of pharmaceutical blister packaging plastic material thermoformed to the desired three-dimensional shape. This layer forms special cavities which serve as a rigid base for the sample chamber (2), wash chamber, secondary fluid and waste chamber and micro-fluidic channels (9). Flexible cover layer (12) is cut to the desired shape from single or multi-layer medical packaging film or flexible metal foils. The reagent pad, wash buffer and secondary fluid (chemiluminescent enzyme substrate) are encapsulated in the fluid chambers between a thermoformed cartridge base layer (17) and the flexible cover layer (12).

The porous membrane element is completely encapsulated from the top, bottom and sides between membrane base encapsulation layer (13) and membrane top encapsulation layer (14) and forms detection channel within encapsulating plastic matrix. It also have fluid inlet and outlets (16) through the encapsulating matrix connected detection channel to micro-fluidic channels. All of the above mentioned layers are either sealed or heat sealed together with intermediate binding layers (not shown in the figures).

In the embodiment of the device wherein the pressure is applied to the sample chamber, secondary chamber and wash chamber by an air pressure application port, the air pressure can be applied to the device through one or more air pressure application ports.

To gain a better understanding of the invention described herein, the following examples are set forth. It should be understood that these examples are for illustrative purposes only. Therefore, they should not limit the scope of this invention in any way.

### EXAMPLES

### Example 1

### Detection of Biotin-Labeled Alkaline Phosphatase

Porous membrane strip with analyte capture zone was prepared using following procedure. Porous nylon membrane Immunodyne™ ABC (5 micron pore size) sheet was printed with 1.5 mm wide line of 5 mg/ml Streptavidin solution in phosphate buffer. Streptavidin was immobilized inside of the porous membrane capture zone by covalent binding to the membrane surface. The porous membrane sheet was further treated with Superblock blocking solution from Pierce Scientific. The porous membrane sheet was cut into 2 mm wide strips after drying. Porous membrane strips were encapsulated between two layers of thermoplastic material forming detection channel. An encapsulated porous membrane strip was mounted on the micro fluidic cartridge by double-sided adhesive pressure sensitive binding layer.

The assay was initiated by placing different volumes of the 1 pM biotin labeled Alkaline Phosphatase (b-AP) sample into a syringe. The syringe was then inserted into a positive displacement syringe pump and connected to a sample application port on the sample chamber on the cartridge through a flexible pipe attached by a pressure tight clamp. As the sample moved along the porous membrane strip by capillary forces, additional external pressure was applied via the syringe pump. Pressure applied through the positive displacement syringe pump resulted in 1, 10 and 100 microliters sample volume pushed through the porous membrane strip analyte capture zone. It took only 90 seconds to push 10 microliters of sample through the membrane pores and 15 minutes to push 100 microliters of sample. For comparison, it took 15 min for the lateral flow membrane (LFM) to absorb 10 microliters of sample by capillary force.

Labelled b-AP was captured in the detection channel by the analyte capture zone through a biotin label. The b-AP simulated antibody conjugates complex capture on the membrane and light generation reactions occur. Chemiluminescent (CL) signal was detected with a custom built luminometer. The luminometer is a light-tight box with photodiode and cartridge holder with pressure tight connectors or clamps. The secondary fluid chamber was pre-filled with chemiluminescent substrate. The cartridge was inserted into the holder and the chemiluminescent substrate was injected into the porous membrane strip by applying 30 psi of external pressure to the secondary fluid chamber inlet hole the air pressure port. The change in the luminometer output signal was proportional to the amount of light generated in the analyte capture zone.

Ultra-sensitive substrates were used for the signal generation. Five microlitres of chemiluminescent AP dioxetane substrate from Michigan Diagnostics (10 times concentrated) was injected into the porous membrane through the micro-channel system. This step combined substrate injection and wash steps together (two in one). The front portion of buffered substrate pushes unbound b-AP enzyme along the detection channel of the LFM and out of the analyte capture zone until it became invisible for the light detector. The later portion of buffered substrate reacted with the captured b-AP to generate light, which was detected by the photo diode.

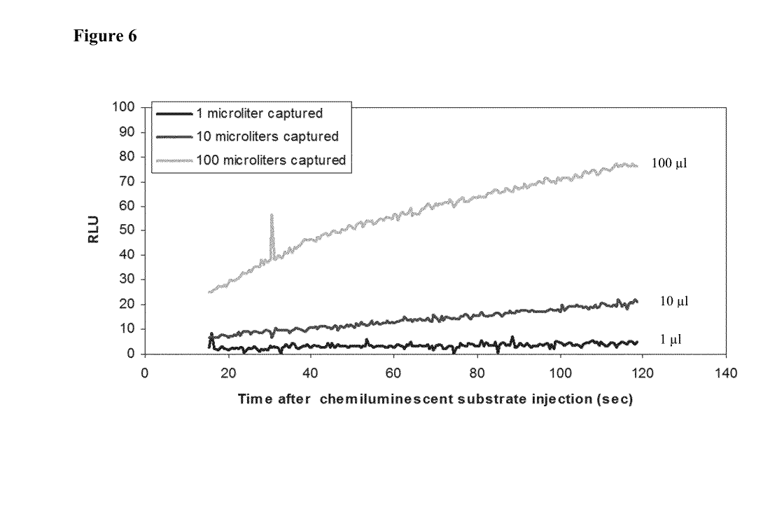

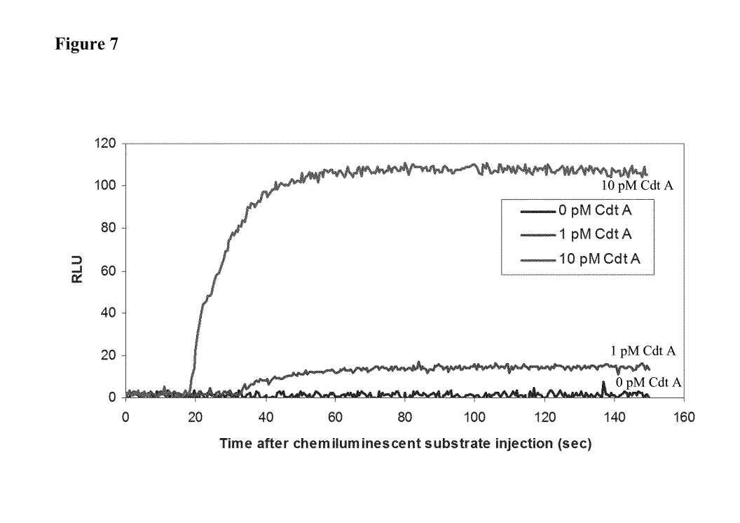

The amount of light generated from the CL signal is proportional to the concentration of analyte in the sample, as well as the volume of sample injected onto the porous membrane. The sensitivity of the assay can be increased dramatically by an increase in the sample volume. The light emission kinetics for variable sample volume with biotinylated AP enzyme is shown in FIG. 6. FIG. 6 demonstrates the effect of the sample volume on light generation and the luminometer response. The volume response data demonstrates pressure assisted assay sensitivity up to 100 times higher than a standard capillary flow assay.

### Example 2

### Detection of Clostridium difficile Toxin a

One example of an assay that can benefit from higher sensitivity is an assay for Clostridium difficile (C. difficile). The bacteria C. difficile is the leading cause of hospital-acquired diarrhea in the United States. (Wilkins, T. D., and D. M. Lyerly. 2003 J. Clin. Microbiol. 41:531-534) The estimated number of cases of C. difficile exceeds 250,000 per year with total additional health care costs approaching US $1 billion annually (Kyne, L., et al. 2002 Clin. Infect. Dis. 34:346-353.)

There are a variety of tests available for the laboratory confirmation of C. difficile. (See Ticehurst, J R et al. 2006 J Clin Microbiol 44:1145-49; Stamper P D et al. 2009 J Clin Microbiol 47:373-8; Kvach E J et al. 2009 J Clin Microbiol October 2009; Eastwood K, et al. 2009 J Clin Microbiol 47:3211-7; and Terhes G, et al. 2009 J Clin Microbiol 47:3478-81.). These are listed in Table 1 and include enzyme immunoassays, both solid phase and rapid immunochromatographic card tests; cell culture cytotoxicity neutralization assays; anaerobic culture; tests for glutamate dehydrogenase (GDH) antigen followed by a toxin test (two-step algorithm); and most recently, nucleic acid amplification testing. The sensitivities of these methods are quite variable (also shown in Table 1) and can be as low as 65% especially when compared to an anaerobic culture method, and some of the assays also have very poor specificity.

TABLE 1
Published Performance of Various C. difficile Testing Methods

| Method | Sensitivity (%) | Specificity (%) |
|---|---|---|
| Enzyme Immunoassays | 33-97 | 83-100 |
| Cell culture neutralization | 65-80 | 97-98 |
| Glutamate dehydrogenase* paired with | 80-98 | 96-98 |
| toxin testing (2-step algorithm) | | |
| Anaerobic toxigenic culture | >90 | 96-97 |
| Nucleic acid amplification | 88-96 | 94-100 |

The device used in this experiment was the device as described in Example 1. The assay was initiated by placing 10 μl of the sample with CdtA onto a reagent pad. The reagent pad contained dried primary biotin labelled anti Clostridium difficile toxin A (CdtA) antibody from Oxoid and secondary horseradish peroxidase (HRP) labelled chicken CdtA specific antibody from Gallus Immunotech. The reagent pad also contained release agents and stabilizers such as Bovine Serum Albumine, Trehalose and Tween 20. The conjugate release matrix was made from Standard-17 glass fibre material from Millipore.

The sample was incubated with the specific antibody conjugates for 10 minutes with heating to 37° C. to form the analyte-antibody complex. The sample was pushed along the porous membrane strip in the detection channel by capillary forces with additional pressure applied by the positive displacement syringe pump with the total transition time of 2 min. The immobilized streptavidin captured the analyte-labelled antibody complex on the porous membrane at the analyte capture zone through the biotin-labelled primary antibody.

A wash step greatly enhanced the sensitivity of the assay by removing unbound HRP conjugate. Fifteen microliters of washing buffer was pushed from the wash chamber into the porous membrane strip through the micro channel system. This step removes free HRP conjugate. The cartridge was pre-filled with chemiluminescent substrate. The cartridge was then inserted into the custom built luminometer (see Example 1) and the 10 microliters of chemiluminescent substrate was injected into the porous membrane strip by applying external pressure to the secondary fluid chamber through the luminometer pressure port. Light generated in the analyte capture zone is directly proportional to the amount of HRP captured. The change in the luminometer output signal is proportional to the amount of light generated in the analyte capture zone, and is directly proportional to the amount of the CdtA toxin in the sample. Ultra-sensitive substrates such as Femtoglow from Michigan Diagnostics or TMA-6 substrate from Limigen were used for the signal generation.

Four microliters of the sample with 0, 1 and 10 pM CdtA pass through the reactor capture zone. According to the dose response data, the analyte detection limit was about 0.4E-18 or 0.4 attomoles of CdtA, which is comparable with standard laboratory methods. FIG. 7 demonstrates the effect of the C. difficile toxin A (CdtA) concentration on light generation and Luminometer response. The light emission kinetics are shown for a Lumigen TMA-6 HRP substrate for variable C. difficile toxin A concentrations in the test sample. The data in FIG. 7 demonstrates very fast substrate kinetics. The signal reaches the steady-state level within about 40 sec after the beginning of injection. A distinct difference in light generation between 0 and 1 pM analyte concentration was measured by Luminometer.

### Example 3

### Detection of HIV p24

An immunoassay cartridge assembled was constructed of plastic films thermoformed to the desired shape, with flexible cover layers and binding adhesive films layers cut to the specific shape. The rigid cartridge forming layer was thermoformed to the specific three-dimensional shape from pharmaceutical blister packaging multiplayer film PVOC 50200 from Teckni-Films with oxygen and water vapour barrier properties. The flexible cover layer was made with medical packaging aluminum foils PS-PET A from Tekni-Films.

The cartridge comprised a wash solution chamber filled with the 100 microliters of washing buffer and a secondary fluid chambers filled with 100 microliters of pre-mixed ready to use TMA-6 chemiluminescent HRP enzyme substrate from Lumigen. Liquids were encapsulated into the sealed compartments for long-term storage. The porous membrane element was encapsulated into the cartridge between plastic layers and formed a flow pass (detection channel) within encapsulating plastic matrix. The porous membrane was made with Immunodyne ABC, 3 micron Nylon porous membrane. Murine monoclonal antibodies against HIV p24 antigen from Zeptometrix were covalently bound to membrane capture site. Porous membrane strips were prepared in the way described in Example 1.

A sample solution with known concentration of HIV p24 antigen was incubated with detecting HRP labeled murine monoclonal antibodies from Immuno Diagnostics. The sample was incubated for 15 min at 37° C. in a vial outside of cartridge. The assay was initiated by placing 35 μl of the pre-incubated sample into the sample entry port.

Negative pressure was applied to the sample waste collector outlet, and the sample was pulled through the porous membrane strip by negative pressure applied downstream the detection channel. Primary analyte specific antibodies immobilized in the analyte capture zone captured the formed HIV p24 antigen-detecting antibody HRP conjugate complex. Assay sensitivity was greatly enhanced by heating the detection channel with porous membrane to 37° C., thus accelerating the analyte capture kinetics.

The sensitivity of assay was further enhanced by the removal of unbound HRP labelled detecting antibodies conjugate. 15 μl of washing buffer followed by 10 μl of TMA-6 chemiluminescent substrate was pushed into the porous membrane strip through the micro channel system by applying mechanical pressure to the flexible cover of the fluid chambers.

Light generated in the analyte capture zone by the chemiluminescent substrate was proportional to the amount of HIV p24 analyte captured on the analyte capture zone. Reacted cartridges were placed into a dark, light impermeable box to obtain images of the detection channel with the analyte capture zone using a charge-coupled device (CCD camera). Light emitting capture zone was observed on CCD images of the cartridge taken with 1-60 seconds exposure. In presence of p24 analyte in the sample, the analyte capture zone was observed as a sharp and bright line across the detection channel. This line was observed in the analyte capture zone for sample HIV p24 analyte concentration in the range 100 pg/ml to 1 ng/ml. A less bright but still sharp line was observed for sample HIV p24 concentration as low as 10 pg/ml.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A device for detecting an analyte of interest in a sample, wherein the device is a microfluidic cartridge comprising:
a sample chamber for receiving the sample;
at least one secondary fluid chamber;
at least one detection channel fluidly connected to said sample chamber and said at least one secondary fluid chamber via one or more microfluidic channels, wherein each of said at least one detection channels comprises an encapsulated porous membrane strip having at least one analyte capture zone comprising a plurality of immobilized capturing species; and
means for generating a dynamic pressure between the sample chamber and the at least one detection channel to facilitate fluid to flow from the sample chamber through each of the at least one detection channels and to increase the volume of fluid that can flow through each of the at least one detection channels over the volume of fluid that can flow through each of the at least one detection channels when no dynamic pressure is applied.

2. The device of claim 1, wherein the sample chamber and/or the secondary fluid chamber has a flexible cover, flexible bottom and/or flexible walls.

3. The device of claim 1, wherein the sample chamber and/or the secondary fluid chamber is formed by a rigid base layer of the cartridge.

4. The device of any one of claims 1-3, wherein the dynamic pressure is generated by pressure applied to the sample chamber.

5. The device of claim 4, wherein the pressure applied is applied to the sample chamber by a mechanical actuator.

6. The device of claim 4 or 5, wherein the pressure applied is between about 5 psi and about 300 psi, or between about 5 psi and 100 psi.

7. The device of any one of claims 1-6, wherein the detection channel is formed with adhesive or thermoplastic film with high physical adhesion to the porous membrane.

8. The device of any one of claims 1-3, wherein the dynamic pressure is generated by a vacuum applied to the downstream end of the detection channel.

9. The device of any one of claims 1-8, wherein the secondary fluid chamber is connected to the microfluidic channel.

10. The device of any one of claims 1-9, further comprising a waste chamber at the downstream end of the detection channel.

11. The device of claim 10, wherein the waste chamber comprises a stop flow valve.

12. The device of claim 11, wherein the stop flow valve is covered by a membrane which is permeable to gas and impermeable to fluid.

13. The device of claim 11 or 12, wherein the stop flow valve has an adaptor to receive a vacuum.

14. The device of any one of claims 1-13, wherein the sample chamber comprises a reagent pad.

15. The device of claim 14, wherein the reagent pad comprises analyte specific antibodies, enzyme labelled antibody conjugates, or optionally labelled capture species.

16. The device of claim 14 or 15, wherein the reagent pad comprises release agents and/or stabilizers.

17. The device of any one of claims 1-16, wherein the immobilized capturing species is an antibody that can specifically bind the analyte of interest.

18. The device of any one of claims 1-16, wherein the immobilized capturing species is streptavidin.

19. The device of any one of claims 1-18, comprising more than one detection channel.

20. The device of any one of claims 1-19, wherein the detecting channel is formed between layers of the cartridge.

21. The device of any one of claims 1-20, wherein the secondary fluid chamber is pre-filled with washing buffer.

22. The device of any one of claims 1-21, wherein the secondary fluid chamber is pre-filled with enzyme substrate.

23. The device of claim 22, wherein the enzyme substrate is a chromogenic, a fluorescent or a chemiluminescent substrate.

24. The device of any one of claims 1-23 comprising more than one secondary fluid chamber.

25. The device of any one of claims 1-24, wherein the cartridge is disposable.

26. The device of any one of claims 1-25, wherein the sample chamber, the detection channel or both can be heated to a predetermined temperature or temperature range.

27. The device of claim 26, wherein the predetermined temperature or temperature range is from about 25° C. to about 50° C. or about 37° C.

28. The device of any one of claims 1-27, wherein the volume of fluid flowing through the detection channel when the dynamic pressure is applied is 2 to 200 times, and preferably 10 times greater than the volume of fluid flowing without the applied dynamic pressure.

29. The device of any one of claims 1-28, wherein the cartridge is made from the two or more layers of pharmaceutical blister packaging materials and/or plastic molded parts.

30. The device of claim 29, wherein the pharmaceutical blister packaging materials comprise metal foils or multi-layer plastic films.

31. The device of any one of claims 1-30, wherein the porous membrane has a pore size from about 0.1-20 microns, and preferably 0.45-5 microns.

32. The device of any one of claims 1-31, wherein the microfluidic channel connects the secondary fluid chamber to the detecting channel.

33. A method of detecting an analyte in a fluid sample, the method comprising:
a) introducing a sample into a sample chamber with a plurality of detecting species under conditions suitable for formation of complexes between the analyte and detecting species;
b) injecting the sample comprising the complexes formed in step (a) onto a porous membrane having an analyte capture zone with a plurality of immobilized capturing species; and
c) detecting the presence of the analyte by detecting complexes immobilized at the analyte capture zone,
wherein the step of injecting the sample is carried out by generating a dynamic pressure between the sample chamber and the detection channel, and
wherein the sample chamber and the detection channel are fluidly connected by a microfluidic channel in a cartridge.

34. The method of claim 33, wherein the dynamic pressure is generated by pressure applied to the sample chamber.

35. The method of claim 33 or 34, wherein the pressure applied is applied to the sample chamber by a mechanical actuator.

36. The method of claim 35, wherein the pressure applied by the mechanical actuator is between about 1 psi and about 300 psi, preferably between about 5 psi and about 100 psi.

37. The method of claim 33, wherein the dynamic pressure is generated by a vacuum applied to the downstream end of the detection channel.

38. The method of any one of claims 33-37, wherein the injecting step takes from between about 1 to 60 minutes, preferably between about 10 and 20 minutes.

39. The method of any one of claims 33-38, wherein the sample comprises water, a water based extract, or a biological fluid.

40. The method of any one of claims 33-39, further comprising the step of
washing unbound detecting species from the capture zone in the detection channel prior to step (c),
wherein the step of washing unbound detecting species comprises generating a dynamic pressure between the secondary fluid chamber and the detection channel.

41. The method of claim 40, wherein the washing step takes between about 10 seconds and about 300 seconds, preferably less than 60 seconds.

42. The method of any one of claims 33-41, wherein the volume of sample flowing through the detection channel when the dynamic pressure is applied is 10 to 100 times greater than the volume of sample flowing through the detection channel in the absence of the applied dynamic pressure.

43. The method of any one of claims 33-42, wherein the cartridge is made from two or more layers of pharmaceutical blister packaging materials and/or plastic molded parts.

44. The method of any one of claims 33-43, wherein the detecting step is carried out using a signal-detecting instrument.

45. The method of any one of claims 33-44, wherein the detecting species comprises a conjugated enzyme and the detecting step comprises injecting enzyme substrate into the detection channel.

46. The method of claim 45, wherein the enzyme converts the enzyme substrate to a detectable product.
